(19) United States
(12) Patent Application Publication
(10) Pub. No.: US 2009/0291025 A1
(43) Pub. Date: Nov. 26, 2009

(54) Microchip And Method Of Using The Same

(75) Inventor: Youichi Aoki, Kyoto-shi (JP)

Correspondence Address: FISH & RICHARDSON P.C., P.O. BOX 1022, MINNEAPOLIS, MN, 55440-1022, US

(73) Assignee: Rohm Co., Ltd., Kyoto (JP)

(21) Appl. No.: 12/467,404

(22) Filed: May 18, 2009

(30) Foreign Application Priority Data
May 20, 2008 (JP) 2008-132135
May 22, 2008 (JP) 2008-134376

(51) Int. Cl. G01N 33/00; B01L 3/00

(52) U.S. Cl. 422/68.1; 422/99; 366/348

(57) ABSTRACT

A microchip is provided, which includes: a second substrate, and a first substrate having a groove on its surface, stacked on the second substrate; and a fluid circuit composed of a cavity defined by the groove and a surface of the second substrate on the side of the first substrate; wherein the fluid circuit includes a mixing portion as a chamber for mixing two or more liquids; and at least a part of a wall forming the mixing portion consists of a linearly extending first wall and a second wall linearly extending from an end of the first wall. A method of using the microchip is also provided.

### BACKGROUND OF THE INVENTION

1. Field of the Invention

The present invention relates to a microchip useful as a μ-TAS (Micro Total Analysis System) used for environmental analysis, chemical synthesis and biochemical test of DNA, protein, cells, immunity, blood and the like as well as to a method of using the microchip.

2. Description of the Background Art

Recently, in the field of medical, health, food and drug discovery, importance of sensing, detecting and estimating quantity of chemical substance and biological matter such as DNA (Deoxyribo Nucleic Acid), enzyme, antigen, antibody, protein, viruses and cells has been increasing, and various biochips and micro chemical chips (hereinafter generally referred to as microchips) allowing easy examination of these have been proposed. The microchip enables a series of experiments/analysis operations, which has been conducted in a laboratory, within a chip having the size of a few centimeters to ten centimeters square and the thickness of a few millimeters to a few centimeters. Therefore, it is advantageous in many aspects. For example, it requires small amount of specimen and reagent, its cost is low, reaction speed is fast and hence inspection with high throughput is possible, and the result of inspection can be provided immediately at the site where the specimen is taken. A microchip is suitably used, for example, for biochemical test such as blood test.

A microchip typically has a “fluid circuit” therein, which is a network of flow paths including portions (chambers) at which specific treatments are done on the fluid, and fine flow paths appropriately connecting these portions (for example, see Japanese Patent Laying-Open No. 2006-300741). A test/analysis of a specimen (in a blood test, blood or specific component contained in blood) using a microchip having therein a fluid circuit as such involves various fluid treatments such as measurement of specimen introduced to the fluid circuit and measurement of liquid reagent to be mixed therewith and mixing of the specimen and the reagent. Such various fluid treatments are done by applying centrifugal force in an appropriate direction to the microchip.

When a specimen or a specific component of the specimen as an object of test/analysis is to be mixed with a liquid reagent in a microchip and the specimen or the specific component is to be treated with the liquid reagent (or to cause reaction with the liquid reagent), the microchip typically has a mixing portion, which is a chamber for bringing together and mixing these liquids, as a part of the fluid circuit. By way of example, Japanese Patent Laying-Open No. 2007-017342 (particularly, FIGS. 8 and 9) discloses a chip having a mixing portion for mixing blood plasma separated from whole blood with a reagent held in a reagent reservoir.

### SUMMARY OF THE INVENTION

The specimen or the specific component of the specimen and the liquid reagent must be fully mixed at the mixing portion. If mixing is insufficient, the treatment to be done on the specimen or the specific component of the specimen by the liquid reagent would be insufficient, or reaction between the specimen or the specific component of the specimen and the liquid reagent would be insufficient, possibly resulting in inaccurate test/analysis.

A first object of the present invention is to provide a microchip including a mixing portion having superior mixing efficiency allowing highly efficient mixing of two or more different types of liquids (typically, a specimen or a specific component of the specimen and one or more liquid reagents) of a relatively simple structure, as well as to provide a method of using the microchip.

Depending on the type of specimen or the specific component of the specimen as the object of test/analysis, two or more different types of liquid reagents may be mixed with the specimen or the specific component of the specimen in the microchip. In such a situation, for appropriately treating the specimen or the specific component of the specimen with the two or more liquid reagents, in some cases, the two or more liquid reagents must be mixed separately with the specimen or the specific component of the specimen.

Therefore, a second object of the present invention is to provide a microchip by which a plurality of different types of liquid reagents can be mixed separately with the specimen or the specific component of the specimen and by which accuracy of test and analysis can further be improved.

The present invention provides a microchip, including: a second substrate, and a first substrate having a groove on its surface, stacked on the second substrate; and a fluid circuit composed of a cavity defined by the groove and a surface of the second substrate on the side of the first substrate; wherein the fluid circuit includes a mixing portion as a chamber for mixing two or more liquids; and at least a part of a wall forming the mixing portion consists of a linearly extending first wall and a second wall linearly extending from an end of the first wall. Preferably, an inner angle formed by the first and second walls is about 90°.

Preferably, the wall defining the mixing portion includes a third wall extending from an end of the first wall opposite to the end coupled to the second wall, and a fourth wall extending from an end of the second wall opposite to the end coupled to the first wall. Preferably, an inner angle formed by the first and third walls and an inner angle formed by the second and fourth walls are each larger than 0° and smaller than 180°.

Further, the present invention provides a method of using the above-described microchip. The method of using the microchip includes: the step of introducing two or more liquids to the mixing portion to obtain a liquid mixture by applying centrifugal force to the microchip; the first mixing promoting step of applying centrifugal force in a direction substantially vertical to the first wall to the microchip; and the second mixing promoting step of applying centrifugal force in a direction substantially vertical to the second wall. Preferably, the first and second mixing promoting steps are repeated alternately.

Further, the present invention provides a microchip, including: a second substrate and a first substrate having a groove on its surface, stacked on the second substrate; and a fluid circuit composed of a cavity defined by the groove and a surface of the second substrate on the side of the first substrate; wherein the fluid circuit includes a first mixing portion for mixing a specimen with a first liquid reagent to prepare a primary liquid mixture, a second mixing portion for mixing the first primary liquid mixture with a second liquid reagent to prepare a secondary liquid mixture, and a third mixing portion for mixing the secondary liquid mixture with a third liquid reagent to prepare a tertiary liquid mixture; at least one of the first, second and third mixing portions includes a residue separating portion for separating residue contained in at least one of the primary, secondary and tertiary liquid mixtures; and the residue separating portion is structured to allow separation of the residue by applying centrifugal force in a first direction to the microchip, and to allow only a supernatant to flow out from the residue separating portion by applying centrifugal force in a second direction.

Preferably, an inner angle θ formed by the first and second directions is 30° to 60° (where θ is an inner angle smaller than 90°).

Preferably, the residue separating portion has an approximately V-shaped wall; and one of two walls forming the approximately V-shaped wall has such a length that prevents the supernatant and the residue from flowing out from the residue separating portion when centrifugal force in the first direction is applied and allows only the supernatant to flow out from the residue separating portion when centrifugal force in the second direction is applied.

Preferably, the fluid circuit further includes a measuring portion for measuring the supernatant flowing out from the residue separating portion.

By the microchip in accordance with the present invention, two or more different liquids, for example, the specimen or the specific component of the specimen and one or more liquid reagents, can be mixed with high efficiency. Therefore, in the microchip, a liquid mixture sufficiently mixed and subjected to appropriate treatment or reaction as intended can be obtained and, hence, highly reliable and accurate test/analysis becomes possible.

Further, by the microchip in accordance with the present invention, it becomes possible to mix a plurality of different types of liquid reagents separately with the specimen or the specific component of the specimen. Further, by the microchip in accordance with the present invention, it is possible to remove residue (solid component or the like) that generates when the specimen or the specific component of the specimen is mixed with the liquid reagent and possibly hinders test/analysis using the microchip, and therefore, accuracy of test and analysis by the microchip can further be improved.

The foregoing and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a top view showing an exemplary structure of a mixing portion provided in a microchip in accordance with a first embodiment of the present invention.

FIG. 2 is a top view showing an exemplary state of the mixing portion shown in FIG. 1 when two or more liquids are introduced by applying centrifugal force.

FIG. 3 is a top view showing an exemplary state of the liquid mixture in the mixing portion shown in FIG. 1, when centrifugal force Y shown in FIG. 1 is applied.

FIG. 4 is a top view showing a preferable example of a microchip in accordance with the first embodiment of the present invention, formed by stacking and joining a first substrate having a groove on its surface to a second substrate.

FIG. 5 is a schematic top view of a preferable example of a microchip in accordance with a second embodiment of the present invention.

FIG. 6 is a schematic top view showing in enlargement a portion of FIG. 5.

FIGS. 7 to 20 are schematic top views showing process steps of an operation of the microchip shown in FIG. 5.

### DESCRIPTION OF THE PREFERRED EMBODIMENTS

### First Embodiment

The microchip in accordance with the present embodiment allows various chemical synthesis, test/analysis and the like using its fluid circuit. In a preferred example, the microchip in accordance with the present embodiment includes a second substrate and a first substrate stacked on and joined to the second substrate. More specifically, on the second substrate, the first substrate having a groove on its surface is joined such that the surface having the groove formed thereon of the first substrate faces the second substrate. Therefore, the microchip consisting of two substrates as such has a fluid circuit composed of a cavity by the groove formed on the surface of first substrate and that surface of the second substrate which faces the first substrate. Though the shape and pattern of the groove formed on the surface of first substrate are not specifically limited, these are determined such that the structure of cavity formed by the groove and the surface of second substrate realizes a desired, appropriate fluid circuit structure. A groove that can form a fluid circuit may be formed also on that surface of the second substrate which faces the first substrate.

In another preferred example, the microchip in accordance with the present embodiment includes a first substrate having a groove on both surfaces of the substrate, and second and third substrates stacked and joined to sandwich the first substrate. The microchip formed of three substrates has two layers of fluid circuits, that is, the first fluid circuit composed of that surface of the second substrate which faces the first substrate and a groove formed on that surface of the first substrate which faces the second substrate, and the second fluid circuit composed of that surface of the third substrate which faces the first substrate and a groove formed on that surface of the first substrate which faces the third substrate. Here, “two layers” mean that fluid circuits are formed at two different positions with respect to the thickness direction of the microchip. The first and second fluid circuits may be coupled by one or more through holes formed penetrating through the first substrate in the thickness direction. A groove that can form a fluid circuit may be formed on that surface of the second and/or third substrate which faces the first substrate.

The method of joining substrates to each other is not specifically limited. By way of example, a joining surface of at least one of the substrates to be joined may be welded (welding), or the substrates may be adhered using an adhesive. Welding may include a method of welding the substrate by heating; welding by irradiating with laser beam or the like, utilizing heat generated by optical absorption; and welding using ultrasonic wave.

The size of microchip in accordance with the present embodiment is not specifically limited, and it may have the width/length of a few centimeters (cm) and the thickness of a few millimeters (mm) to about 1 centimeter (cm).

Materials of substrates forming the microchip in accordance with the present embodiment are not specifically limited. Available examples include: organic materials such as polyethylene terephthalate (PET), polybutylene terephthalate (PBT), polymethylmethacrylate (PMMA), polycarbonate (PC), polystyrene (PS), polypropylene (PP), polyethylene (PE), polyethylene naphthalate (PEN), poly arylate resin (PAR), acrylonitrile-butadiene-styrene resin (ABS), vinyl chloride resin (PVC), polymethylpentene resin (PMP), polybutadiene resin (PBD), biodegradable polymer (BP), cycloolefin polymer (COP), polydimethylsiloxane (PDEM); and inorganic materials such as silicon, glass and quartz.

When the microchip is formed by first and second two substrates, though not specifically limiting, the first substrate having a groove on its surface to be stacked on the second substrate may be a transparent substrate. Thus, a detecting portion formed by the groove of transparent first substrate and the surface of second substrate can be provided as a part of fluid circuit. Using such a microchip having such a detecting portion, it becomes possible to conduct optical measurement, by introducing the object of measurement (for example, a liquid mixture of specimen as the object to be tested/analyzed and a liquid reagent) to the detecting portion, irradiating the detecting portion with light, and detecting intensity (transmittance) of the light transmitted through the mixture. The second substrate may be a transparent substrate, or it may be a colored substrate. For example, the substrate may be a black substrate formed of resin, having carbon black or the like added to the resin. A colored substrate is preferable and black substrate is more preferable. When the second substrate is provided as a colored substrate, welding with laser beam becomes available. When substrates are joined by laser welding, it is the joining surface of the colored substrate that is mainly welded for joining and, therefore, deformation of a groove formed in the transparent first substrate can be minimized.

When the microchip is formed by first to third three substrates, though not specifically limiting, the second and third substrates holding therebetween the first substrate with a groove on both surfaces may be transparent substrates. Thus, a detecting portion formed by the through hole passing through the first substrate in the thickness direction and the surfaces of transparent second and third substrates can be provided as a part of fluid circuit. Using such a microchip having such a detecting portion, it becomes possible to conduct optical measurement, by introducing the object of measurement (for example, a liquid mixture of specimen as the object to be tested/analyzed and a liquid reagent) to the detecting portion, irradiating the detecting portion with light in a direction vertical to the microchip surface from the top surface (or bottom surface) of the microchip, and detecting intensity (transmittance) of the light transmitted through the mixture from the opposite side. The first substrate positioned between the second and third substrates should preferably be a colored substrate and, more preferably a black substrate.

The method of forming a groove (flow path pattern) serving as the fluid circuit is not specifically limited, and injection molding using a metal mold with transfer structure, imprinting and the like may be available. If the substrate is to be formed with inorganic material, etching is also available.

In the microchip in accordance with the present embodiment, the fluid circuit (where two layers of fluid circuits are provided, the first and second fluid circuits) includes various portions arranged at appropriate positions in the fluid circuit to enable various appropriate treatments for the liquid in the fluid circuit, and these portions are appropriately connected through fine flow paths.

In the microchip in accordance with the present embodiment, the fluid circuit is provided with at least a mixing portion, which is a chamber for bringing into contact with and mixing two or more different types of liquids. The fluid circuit may include only one mixing portion or two or more mixing portions. The mixing portion in accordance with the present invention will be described in detail later. The fluid circuit may have a portion other than the mixing portion, of which examples may include: a separating portion for extracting a specific component from specimen introduced to the fluid circuit; a specimen measuring portion for measuring specimen; a liquid reagent holding portion for holding liquid reagent; a liquid reagent measuring portion for measuring liquid reagent; and a detecting portion (cuvette for optical examination) for testing/analyzing liquid mixture (for example, detection or quantification of specific component in the liquid mixture) obtained by mixing the specimen and the liquid reagent. The microchip in accordance with the present embodiment may have all or some of these portions listed as examples. Further, the microchip may have a portion or portions other than those mentioned above. These portions are arranged at appropriate positions in the fluid circuit and connected through fine flow paths so as to enable desired fluid treatment. Each of these portions may be provided in plural numbers.

In the present invention, the “liquid reagent” refers to liquid substance to be mixed with the specimen as the object of test/analysis, for treating the specimen or to cause reaction with the specimen, for the test/analysis using the microchip. The microchip may contain only one liquid reagent, or it may contain two or more liquid reagents. The “specimen” refers to the substance itself (for example, blood) as the object of test/analysis introduced to the fluid circuit, or a specific component (for example, blood plasma, blood cell component) contained in the substance.

When the microchip in accordance with the present embodiment is composed of two substrates (first and second substrates), typically, the microchip in accordance with the present embodiment has a liquid reagent inlet for introducing the liquid reagent to the liquid reagent holding portion, as a pass-through inlet reaching the liquid reagent holding portion (passing through the first substrate in its thickness direction) formed on the upper surface (that is, the surface of first substrate). Typically, liquid reagent is introduced through the liquid reagent inlet, then a label or sticker for sealing the liquid reagent inlet is adhered on the surface of microchip (the surface of first substrate), and the microchip is ready for use. When the microchip is composed of three substrates (second substrate/first substrate/third substrate), the liquid reagent inlet may be provided as a pass-through inlet that passes through the second or third substrate in its thickness direction.

Further, the microchip in accordance with the present embodiment generally has a specimen introducing inlet, through which the specimen is introduced to the fluid circuit. The specimen introducing inlet may be formed as a through hole passing from one surface of microchip to the fluid circuit. By way of example, it may be formed as a sample tube placing portion, which is a through hole extending from one surface of the microchip to the fluid circuit for incorporating a sample tube such as a capillary containing the specimen. Specifically, when the microchip is composed of the first substrate having a groove on the substrate surface and the second substrate, the specimen introducing inlet may be formed as a pass-through hole passing through the first substrate in its thickness direction. Further, if the microchip is formed by joining the third substrate, the first substrate having a groove provided on opposite surfaces and the second substrate in this order, the specimen introducing inlet may be formed as a pass-through hole passing through the third substrate (or the second substrate) in its thickness direction. The separating portion is connected to the specimen introducing inlet through a flow path, and hence, the specimen introduced from the specimen introducing inlet can be guided to the separating portion.

The liquid mixture finally obtained by mixing the specimen with one or more liquid reagents is subjected to an optical measurement or the like for test/analysis, by a method of irradiating the portion (for example, the detecting portion) containing the liquid mixture with light and detecting intensity (transmittance) of transmitted light, though the method is not limiting.

Various fluid treatments that take place in the fluid circuit including extraction of specific component from specimen (separation of unnecessary component), measurement of specimen and/or liquid reagent, mixing of specimen and liquid reagent, and introduction of resulting liquid mixture to the detecting portion can be realized by successively applying centrifugal force in appropriate directions to the microchip. The centrifugal force is applied to the microchip by mounting the chip on an apparatus that can impart centrifugal force to the chip (centrifugal device). The centrifugal device includes a rotor that can freely rotate about a centrifugal axis, and a freely rotatable stage arranged on the rotor. By placing a microchip on the stage, rotating the stage to set an angle of microchip with respect to the rotor as desired, and rotating the rotor about the centrifugal axis, it is possible to apply centrifugal force in a direction as desired to the microchip.

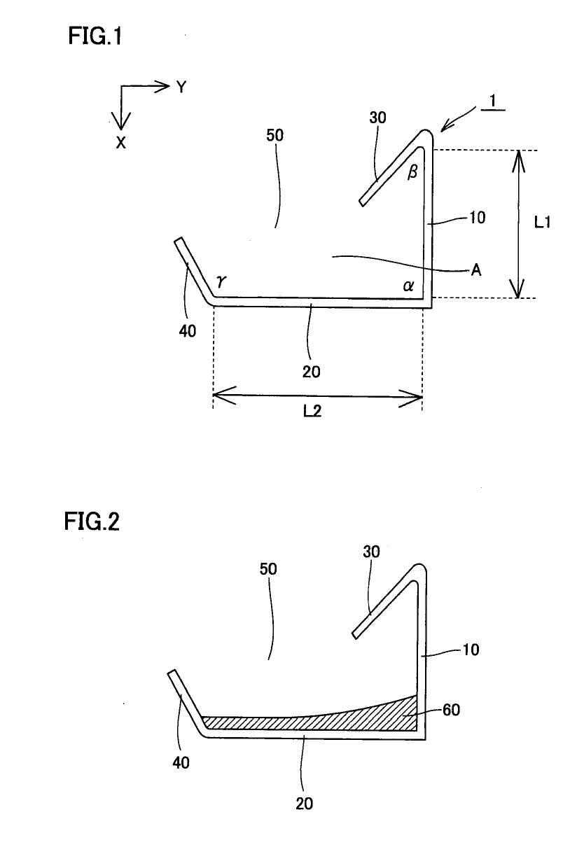

Next, the mixing portion of microchip in accordance with the present embodiment will be described. FIG. 1 is a top view showing an exemplary structure of mixing portion provided in the microchip in accordance with the present embodiment. FIG. 1 is a top view from the side of first substrate, of a microchip having a fluid circuit therein formed by joining a first substrate having a groove on its surface and a second substrate, showing in enlargement a region where the mixing portion is positioned. The fluid circuit formed by the groove on the surface of first substrate and the surface of second substrate is actually formed inside the microchip and, therefore, walls defining the mixing portion are also formed inside the microchip. In FIG. 1, however, the walls are indicated by solid lines.

Mixing portion 1 shown in FIG. 1 as a part of fluid circuit includes first to fourth walls 10, 20, 30 and 40. In an area A partitioned by these four walls, two or more liquids are mixed. Such an area A corresponds to the bottom surface of the groove formed on the surface of first substrate.

The first wall 10 has a linear (linear or substantially linear) shape, and the second wall 20 extends linearly from one end of first wall 10 to a direction different from first wall 10. Further, the wall defining mixing portion 10 further includes the third wall 30 extending linearly from an end of first wall 10 opposite to the one end coupled to the second wall 20, and the fourth wall 40 extending linearly from an end of second wall 20 opposite to the one end coupled to the first wall 10.

At mixing portion I consisting of a chamber partitioned by the first to fourth walls 10, 20, 30 and 40, two or more different types of liquids can be mixed with high efficiency. The two or more liquids may be introduced from an opening 50 at an upper side of mixing portion 1 in FIG. 1 to the area A of mixing portion 1, and the liquid mixture resulting from the mixing operation can also be discharged from mixing portion 1 through opening 50.

Mixing of liquids at mixing portion 1 will be described in detail. First, two or more different types of liquids to be mixed are introduced to area A of mixing portion 1. The two or more different types of liquids to be mixed include, though not specifically limiting, a specimen (or a specific component in the specimen) as an object of test/analysis using the microchip, and a liquid reagent for treating or to cause reaction with the specimen. Here, two or more liquid reagents may be mixed with the specimen. The two or more different types of liquids are introduced to mixing portion 1 by applying to the microchip centrifugal force with downward component of FIG. 1 (for example, centrifugal force X shown in FIG. 1: the direction of centrifugal force X is vertical to the second wall 20).

FIG. 2 shows an exemplary state where two or more different types of liquids are introduced through opening 50 to mixing portion 1, by the application of centrifugal force with downward component of FIG. 1. The two or more liquids (liquid mixture 60) introduced from opening 50 and put together are pressed to the inner wall surface of second wall 20 and spread entirely over the inner wall surface, and extend along the linear inner wall surface of second wall 20. In the state shown in FIG. 2, liquid mixture 60 does not uniformly spread over the inner wall surface of second wall 20 but spreads slightly to the right (closer to the first wall 10), because the force (centrifugal force) received by liquid mixture 60 has not directly downward but slightly rightward component. Such a phenomenon occurs, for example, when mixing portion 1 is arranged, on the microchip plane, slightly to the right from the central position of microchip and the centrifugal force applied to the microchip at the center of microchip has downward direction.

In the mixing portion in accordance with the present embodiment, it is preferred to provide the fourth wall 40 continuous to the second wall 20 as in mixing portion 1 shown in FIG. 1. When liquid is introduced to the mixing portion, the fourth wall 40 prevents the liquid from leaking and being discharged from the mixing portion at the end of second wall 20 opposite to the side of first wall 10. To attain this function, the inner angle formed by the second and fourth walls 20 and 40 (angle γ of FIG. 1) should preferably be larger than 0° and smaller than 180° and, more preferably, from 45° to 135°.

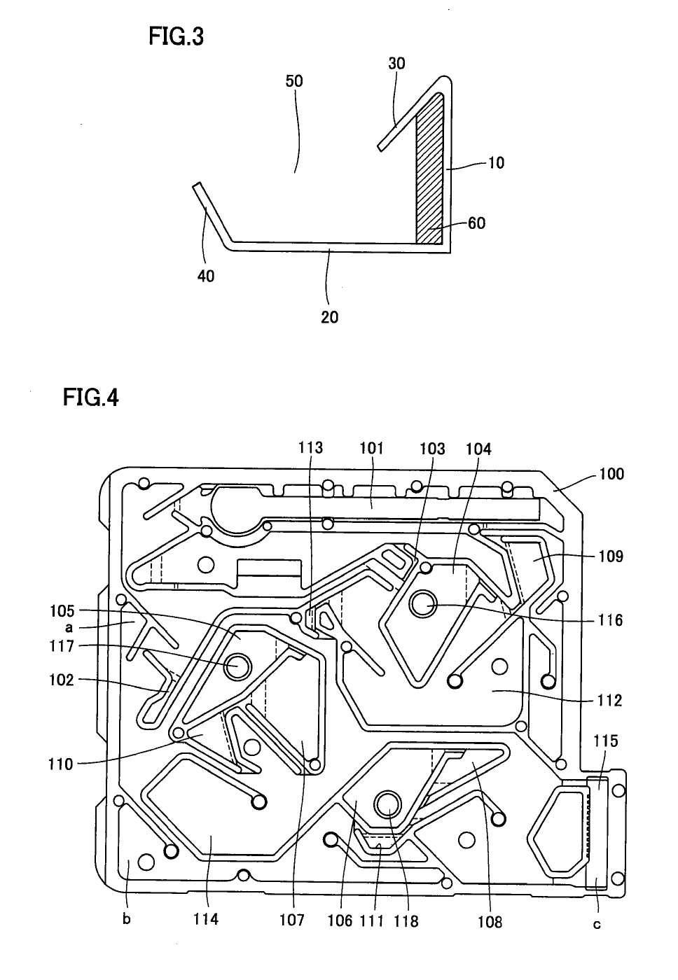

Next, centrifugal force with rightward component of FIG. 1 (for example, centrifugal force Y shown in FIG. 1: the direction of centrifugal force Y is vertical to the first wall 10) is applied to the microchip (first mixing promoting step). FIG. 3 shows an exemplary state of liquid mixture in mixing portion when centrifugal force Y shown in FIG. 1 is applied. By the application of such centrifugal force, liquid mixture 60 is pressed to the inner wall surface of first wall 10 and spread entirely over the inner wall surface, and extend along the linear inner wall surface of first wall 10. More specifically, the liquid mixture that has been spread over the inner wall surface of second wall 20 is once collected and contracted at or near the inner angle portion formed by the first and second walls 10 and 20, and then spread again over the inner wall surface of first wall 10 because of the centrifugal force. As the liquid mixture is expanded/contracted, mixing of liquids is efficiently promoted. Though the direction of centrifugal force at this stage is not necessarily vertical to the first wall 10, substantially vertical direction is preferred.

In the mixing portion in accordance with the present embodiment, it is preferred to provide the third wall 30 continuous to the first wall 10 as in mixing portion 1 shown in FIG. 1. When the liquid mixture 60 is moved to the inner wall surface of first wall 10, the third wall 30 prevents the liquid mixture 60 from leaking and being discharged from the mixing portion at the end of first wall 10 opposite to the side of second wall 20. To attain this function, the inner angle formed by the first and third walls 10 and 30 (angle β of FIG. 1) should preferably be larger than 0° and smaller than 180° and, more preferably, from 45° to 135°.

Next, centrifugal force with downward component of FIG. 1 (for example, centrifugal force X shown in FIG. 1: the direction of centrifugal force X is vertical to the second wall 20) is again applied, to attain the state similar to the state of FIG. 2 (second mixing promoting step). By the application of such centrifugal force, the liquid mixture that has been spread over the inner wall surface of first wall 10 is once collected and contracted at or near the inner angle portion formed by the first and second walls 10 and 20, and then spread again over the inner wall surface of second wall 20 because of the centrifugal force. This further promotes mixing.

As described above, because of the mixing portion formed to include at least first and second walls 10 and 20, efficient mixing of two or more different types of liquids becomes possible, utilizing contraction/expansion of liquids caused by the application of centrifugal force in different directions. The first and second steps of promoting mixing described above may be repeated alternately. Then, liquid mixture of higher uniformity can reliably be obtained.

The inner angle (angle a of FIG. 1) formed by the first and second walls 10 and 20 is preferably larger than 0° and smaller than 180°, more preferably from 60° to 120° and most preferably 90° or approximately 90°, from the view point of attaining more efficient contraction/expansion of liquids. Further, the length (L1 of FIG. 1) of inner wall surface of first wall 10 and the length (L2 of FIG. 1) of inner wall surface of second wall 20 should preferably be about 10 to about 20 mm, to realize efficient contraction/expansion of liquids.

Various modifications may be made to the mixing portion in accordance with the present embodiment within the scope of the present invention. By way of example, referring to the mixing portion 1 shown in FIG. 1, at the end of third wall 30 opposite to the end coupled to the first wall 10, and/or at the end of fourth wall 40 opposite to the end coupled to the second wall 20, fifth wall and/or sixth wall may be coupled. For example, assume that the fourth wall 40 is formed such that the angle y with the second wall 20 exceeds 90°. Here, if it becomes necessary to apply centrifugal force in the leftward direction of FIG. 1 while liquid mixture is held in the mixing portion, unintended leakage of the liquid mixture occurs from the mixing portion by applying this leftward centrifugal force. By providing a wall continuous to the fourth wall 40 such that the inner angle with the fourth wall 40 is larger than 0° and smaller than 180°, such a problem can be avoided.

Further, opening 50 may be divided into two or more openings by forming a wall. The two or more openings may be used as inlets for introducing two or more liquids to the mixing portion, or as outlets for the resulting liquid mixture. The two or more liquids (for example, specimen and liquid reagent) may be introduced through different openings to the mixing portion.

FIG. 4 is a top view showing a preferred example of the microchip in accordance with the present embodiment, formed by stacking and joining a first substrate 100 having a groove on its surface on a second substrate (not shown). In the microchip shown in FIG. 4, the first substrate 100 is stacked on the second substrate (not shown) with the surface having a groove formed on first substrate 100 facing the second substrate. Specifically, FIG. 4 shows the surface of first substrate 100 opposite to the surface having the groove formed thereon. For convenience of description, however, the groove pattern is shown in solid lines. In the microchip in accordance with the present example, the second substrate has the same or similar contour as the first substrate 100. The first substrate and second substrate are transparent and black plastic substrates, respectively, for example.

Referring to FIG. 4, the microchip in accordance with the present example mainly includes: a sample tube placing portion 101 for incorporating a sample tube such as a capillary containing whole blood taken from a subject; a separating portion 102 for separating the whole blood drawn out from the sample tube to blood cell component and plasma component; a blood cell measuring portion 103 for measuring the separated blood cell component; three liquid reagent holding portions 104, 105 and 106 for holding liquid reagents; liquid reagent containing portions 107 and 108 for temporarily containing liquid reagents, which are provided adjacent to liquid reagent holding portions 105, 106, respectively; three liquid reagent measuring portions 109, 110 and 111 for measuring liquid reagents; a first mixing portion 112 for mixing the blood cell component and the liquid reagent; a liquid mixture measuring portion 113 for measuring the liquid mixture of blood cell component and liquid reagent; a second mixing portion 114 for mixing the liquid mixture of blood cell component and the liquid reagent with another liquid reagent; and a detecting portion 115 for testing/analyzing the finally resulting liquid mixture. The three liquid reagent holding portions 104, 105 and 106 have liquid reagent inlets 116, 117 and 118 for introducing liquid reagents to the corresponding liquid reagent holding portions, respectively. The liquid reagent inlets 116, 117 and 118 are through holes passing through the first substrate 100 in the thickness direction. In the following, the liquid reagents introduced through liquid reagent inlets and held in liquid reagent holding portions 104, 105 and 106 will be referred to liquid reagents R0, R1 and R2, respectively. The first mixing portion 112 has substantially the same structure as mixing portion 1 shown in FIG. 1 described above.

As described above, the microchip in accordance with the present example has a fluid circuit of which structure is suitable for mixing blood cell component separated from the whole blood with liquid reagents R0, R1 and R2 in this order and for conducting test/analysis such as optical measurement, on the resulting liquid mixture.

The method of operating microchip shown in FIG. 4 will be briefly described in the following. The operating method described below is only an example and not limiting. First, a sample tube containing sampled whole blood is inserted to sample tube placing portion 101. Then, centrifugal force in leftward direction of FIG. 4 (hereinafter simply referred to as “leftward”; same for other directions) is applied to the microchip, so that the whole blood sample in the sample tube is taken out, and then downward centrifugal force is applied to let the whole blood sample enter separation portion 102, where the plasma component and blood cell component are separated. Next, leftward centrifugal force is applied, to remove the plasma component as the upper layer. Here, the removed plasma component is contained in an area a. Next, downward centrifugal force is applied, so that liquid surface of blood cell component in separating portion 102 is made smooth, and the removed plasma component is moved to an area b. Next, rightward centrifugal force is applied, so that liquid reagent R0 in liquid reagent holding portion 104 is introduced to liquid reagent measuring portion 109 and measured. By this centrifugal force, liquid reagent R1 in liquid reagent holding portion 105 and liquid reagent R2 in liquid reagent holding portion 106 move to liquid reagent containing portions 107 and 108, respectively. Further, by this centrifugal force, the blood cell component in separating portion 102 is introduced to blood cell measuring portion 103 and measured.

Next, downward centrifugal force is applied so that the measured blood cell component and the measured liquid reagent R0 are mixed at the first mixing portion 112, to obtain a liquid mixture. By this centrifugal force, liquid reagent R2 in liquid reagent containing portion 108 is measured in liquid reagent measuring portion 111. Thereafter, rightward, downward, leftward and downward centrifugal forces are applied successively to sufficiently mix the liquid mixture. By the application of this leftward centrifugal force, liquid reagent R1 in liquid reagent containing portion 107 is measured in liquid reagent measuring portion 110. Further, by the final downward centrifugal force, the measured liquid reagent R1 is moved to the second mixing portion 114. The merit of improving mixing efficiency and the like derived from the arrangement of walls forming the first mixing portion 112 are as described with reference to the mixing portion shown in FIG. 1.

Next, leftward centrifugal force is applied so that solid component in the liquid mixture in first mixing portion 112 is centrifuge-separated, and thereafter, upper-leftward and leftward centrifugal forces are applied successively, so that supernatant of the liquid mixture in first mixing portion 112 is introduced to liquid mixture measuring portion 113 and measured. Next, downward centrifugal force is applied, so that the measured liquid mixture and liquid reagent R1 are mixed at the second mixing portion 114. Thereafter, leftward and downward centrifugal forces are applied successively, to sufficiently mix the liquid mixture. While the downward centrifugal force is applied, the measured liquid reagent R2 is positioned in an area c. Next, rightward centrifugal force is applied, so that the liquid mixture is mixed with liquid reagent R2 at detecting portion 115, and then downward centrifugal force is applied to sufficiently mix. Finally, rightward centrifugal force is applied so that the liquid mixture is contained in detecting portion 115, the detecting portion 115 is irradiated with light, and optical measurement such as measurement of transmitted light intensity, is performed.

### Second Embodiment

The microchip in accordance with the present embodiment will be described, with reference to a preferred example. In the following, mainly the characteristic portions of the microchip in accordance with the present embodiment will be described. Other structures are the same as in the first embodiment. A preferred example of the microchip in accordance with the present embodiment is the same as the microchip shown in FIG. 4.

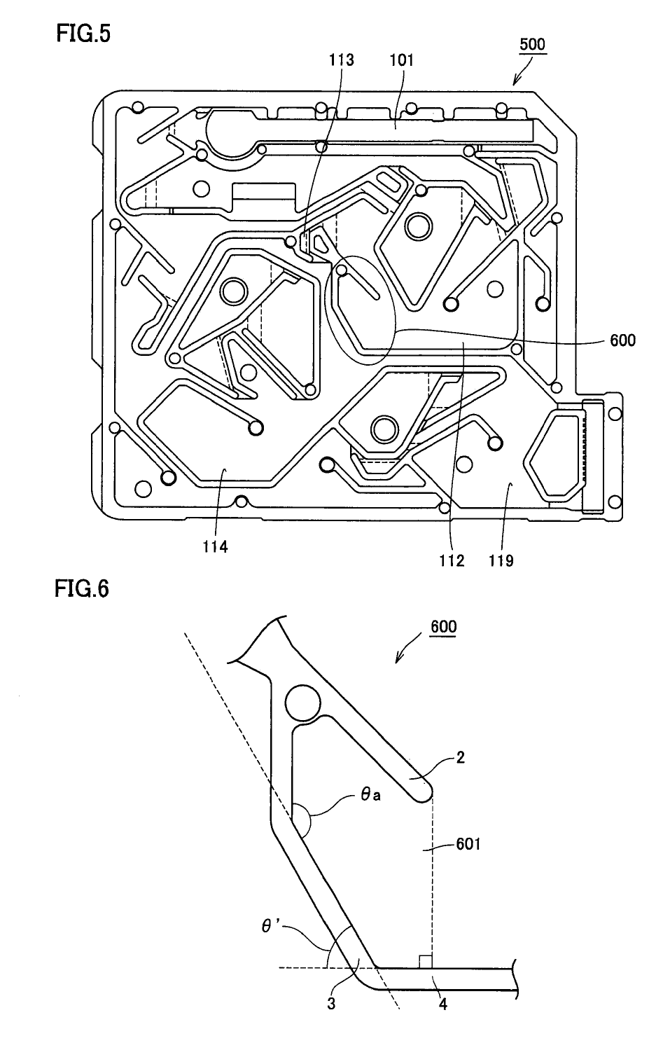

FIG. 5 is a schematic top view showing a preferred example of the microchip in accordance with the present embodiment. FIG. 6 is a schematic top view showing, in enlargement, a portion of FIG. 5.

A microchip 500 shown in FIG. 5 is formed by stacking a second substrate to a surface having a groove thereon of the first substrate, which has a groove forming the fluid circuit on its surface and a through hole passing through its thickness direction. FIG. 5 is a top view showing the surface on the side of first substrate, of such a microchip 500. Actually, the groove forming fluid circuit are formed on the surface opposite to the surface shown in FIG. 5 of the first substrate (the surface to be joined to the second substrate), the groove pattern in shown in solid lines so as to enable clearer understanding of the fluid circuit structure.

The microchip 500 shown in FIG. 5 is 48 to 52 mm long and 38 to 42 mm wide. For example, microchip 500 is held on a stage of a centrifugal device, having the freely rotatable stage mounted on a rotor having a diameter of 45 to 50 cm and freely rotatable about a centrifugal axis. By rotating the rotor about the centrifugal axis, centrifugal force of a desired direction can be applied to microchip 500.

Microchip 500 has a sample tube placing portion 101 for incorporating a sample tube such as a capillary containing a specimen, as a specimen inlet for introducing the specimen to the fluid circuit, which passes through one surface to reach the fluid circuit. Further, the fluid circuit of microchip 500 includes: a first mixing portion 112 for mixing the specimen with a first liquid reagent to prepare a primary liquid mixture; a second mixing portion 114 for mixing the primary liquid mixture with a second liquid reagent to prepare a secondary liquid mixture, and a third mixing portion 119 for mixing the secondary liquid mixture with a third liquid reagent to prepare a tertiary liquid mixture.

The first mixing portion 112 has a residue separating portion 600 for separating residue contained in the primary liquid mixture. In the microchip of the present embodiment, such a residue separating portion can be provided in at least one of the first, second and third mixing portions for separating residue in at least one of the primary, secondary and tertiary liquid mixtures.

Residue separating portion 600 has such a structure that can separate residue contained in the primary liquid mixture when centrifugal force in a first direction is applied to microchip 500. The first direction is, for example, leftward direction of FIG. 5, that is, longitudinal direction of microchip 500. Further, residue separating portion 600 has such a structure that allows only the supernatant of primary liquid mixture to flow out from residue separating portion 600 when centrifugal force in a second direction is applied to microchip 500. The supernatant flowing out from residue separating portion 600 moves to liquid mixture measuring portion 113 and then, the measured supernatant moves to the second mixing portion 114.

As described above, the fluid circuit provided in microchip 500 has such a structure that allows sufficient mixing of the specimen with three different reagents, that is, first, second and third liquid reagents, and allows separation of residue that hinders test and analysis in microchip 500.

Residue separating portion 600 has an approximately V-shaped wall such as shown in FIG. 6. The approximately V-shaped wall is composed of first and second walls 2 and 3. Between the first wall 2 and the second wall 3, an opening 601 is formed. At the end of the second wall 3 opposite to the side coupled to the first wall, a third wall 4 is provided, extending substantially parallel to the first direction.

The first wall 2 forming the approximately V-shaped wall has such a length that prevents leakage of supernatant and residue of primary liquid mixture from residue separating portion 600 when centrifugal force in the first direction is applied and allows leakage of only the supernatant from residue separating portion 600 when centrifugal force in the second direction is applied.

In microchip 500 shown in FIGS. 5 and 6, the first direction may be the leftward direction in FIGS. 5 and 6, though not limiting. Further, the second direction may be the direction diagonally upward by 45° (upper left direction) from the first direction, though not limiting. At the residue separating portion 600, the inner angle θ formed by the first direction as the direction of centrifugal force applied to remove residue of primary liquid mixture and the second direction as the direction of centrifugal force applied to let only the supernatant of primary liquid mixture flow out from the residue separating portion 600 is preferably from 30° to 60°, and more preferably, 40° to 50° (where θ is an inner angle smaller than 90°). This is to prevent part of the residue separated by the application of centrifugal force in the first direction from flowing to the liquid mixture measuring portion 113.

The angle θ′ formed by the second wall 3 and third wall 4 at the connecting portion between the second and third walls 3 and 4 should preferably be 30° to 60° and, more preferably, 45° to 60°. With such an angle, when the centrifugal force is applied in the first direction, it is possible to have the residue and supernatant further away from each other.

The specific structure of the approximately V-shaped wall should appropriately be designed such that the liquid surface of the supernatant of primary liquid mixture is positioned on the left side than a line drawn vertically downward in the first direction from the first wall 2 forming the approximately V-shaped wall.

It is preferred that a portion close to the first wall 2 of second wall 3 forming the approximately V-shaped wall is bent inward to the side of first mixing portion 1 12, as shown in FIG. 6. Specifically, the angle θa formed by the wall closer to the first wall 2 and the wall closer to the third wall 4 of the second wall 3 should preferably be 90° to 180° and, more preferably, 120° to 150°. With such an angle, it becomes easier to force the residue to the direction of application when the centrifugal force in the first direction is applied.

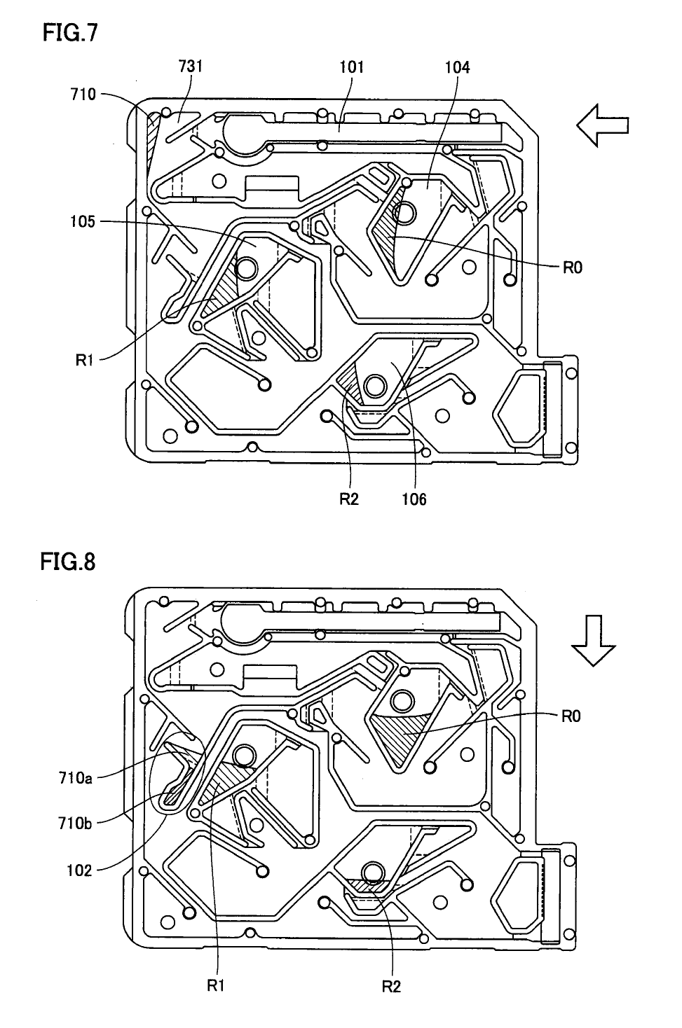

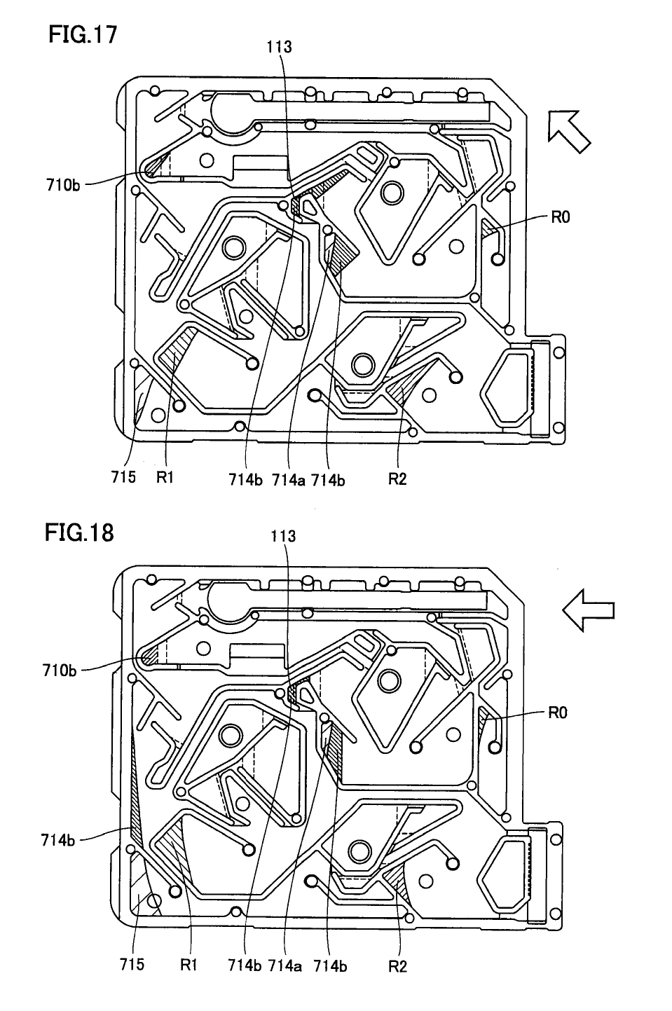

FIGS. 7 to 20 are schematic top views showing operation steps of microchip 500 shown in FIG. 5. FIGS. 16 and 17 show an operation at the residue separating portion 600. In FIGS. 7 to 20, white arrows represent directions of centrifugal force received at the central portion of microchip 500, and the directions will be described as left, right, upward and downward in the figures. Though the operation of microchip 500 will be described taking blood (whole blood) as an exemplary specimen in the following, the specimen is not limited in the present embodiment, and any liquid containing two or more components of different specific gravity may be used as specimen.

First, as shown in Fig, 7, when centrifugal force to the left of FIG. 7 is applied to microchip 500, blood 710 is introduced to specimen introducing portion 731. Liquid reagent R0 is held in liquid reagent holding portion 104, liquid reagent R1 is held in liquid reagent holding portion 105, and liquid reagent R2 is held in liquid reagent holding portion 106.

Next, as shown in FIG. 8, when centrifugal force in downward direction of FIG. 8 is applied to microchip 500, blood 710 is introduced to separating portion 102 and, by the centrifugal separation, it is separated to blood cell component 710b and plasma component 710a having different specific gravities.

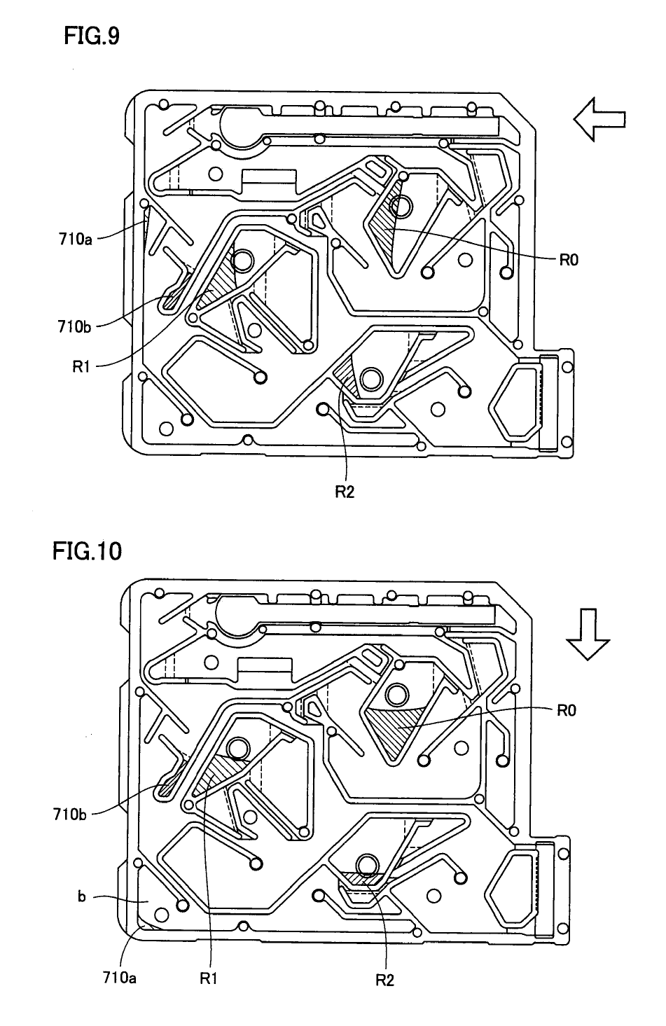

Thereafter, as shown in FIG. 9, when centrifugal force to the left of FIG. 9 is applied to microchip 500, plasma component 710a flows out from separating portion 102. Only the blood cell component 710b is left and held in separating portion 102.

Next, as shown in FIG. 10, by applying centrifugal force in downward direction of FIG. 10 to microchip 500, liquid surfaces of blood cell component 710b and liquid reagents R0, R1 and R2 are made smooth. Application of this downward centrifugal force is performed to introduce plasma component 710a to an excess space b and to feed blood cell component 710b at the next step without any liquid left behind.

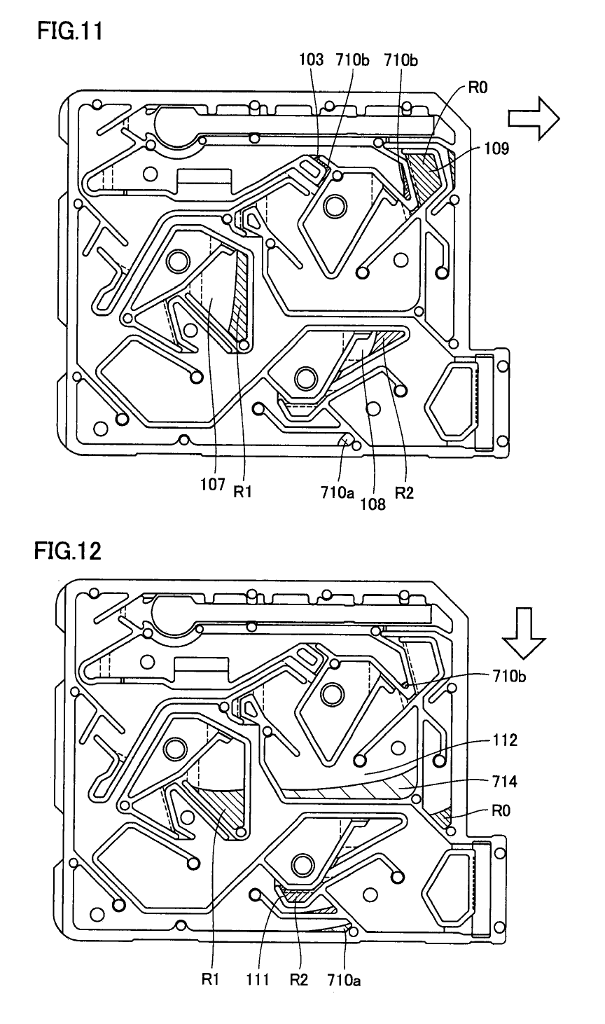

Next, as shown in FIG. 1I, when centrifugal force to the right of FIG. 11 is applied to microchip 500, blood cell component 710b moves from separating portion 102 to blood cell measuring portion 103, and only a prescribed amount is held in blood cell measuring portion 103. Further, liquid reagent R0 moves from liquid reagent holding portion 104 to liquid reagent measuring portion 109, and only a prescribed amount is held in liquid reagent holding portion 109. Further, liquid reagent R1 moves from liquid reagent holding portion 105 to liquid reagent containing portion 107. Further, liquid reagent R2 moves from liquid reagent holding portion 106 to liquid reagent containing portion 108. These liquid reagent containing portions are portions for temporarily holding liquid reagents R1 and R2 before measurement at the liquid reagent measuring portions.

Next, as shown in FIG. 12, by applying centrifugal force in downward direction of FIG. 12 to microchip 500, measured blood cell component 710b and measured liquid reagent R0 are mixed at the first mixing portion 112, and primary liquid mixture 714 is prepared. By this downward centrifugal force, liquid reagent R2 moves from liquid reagent containing portion 108 to liquid reagent measuring portion 111, and only a prescribed amount is held in liquid reagent measuring portion 111.

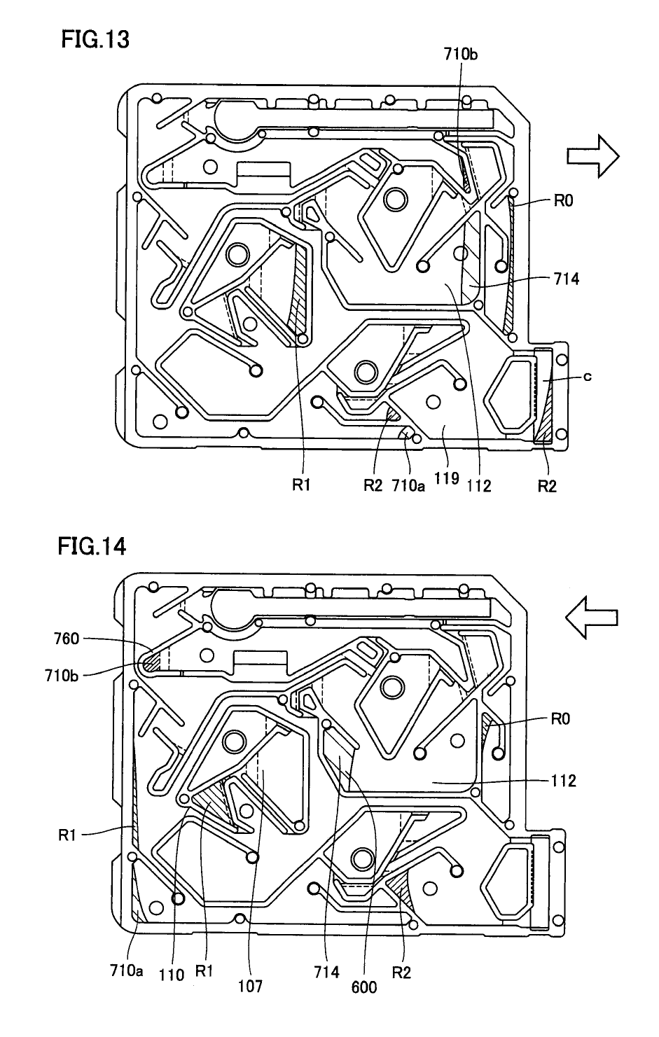

Next, as shown in FIG. 13, by applying centrifugal force to the right of FIG. 13 to microchip 500, primary liquid mixture 714 is sufficiently mixed at the first mixing portion 112. Further, liquid reagent R2 measured at liquid reagent measuring portion 111 moves to the area c of the third mixing portion 119 (the area c also serves as a detecting portion for optical measurement of the liquid mixture), and held therein.

Next, as shown in FIG. 14, when centrifugal force to the left of FIG. 14 is applied to microchip 500, liquid reagent R1 moves from liquid reagent containing portion 107 to liquid reagent measuring portion 110, and only a prescribed amount is held in liquid reagent measuring portion 110. Further, primary liquid mixture 714 is sufficiently mixed as it moves to residue separating portion 600 next to the first mixing portion 112. Further, blood cell component 710b that is not held in blood cell measuring portion 103 moves to an error sensing portion 760. By irradiating error sensing portion 760 with light and measuring transmittance and the like, it is possible to confirm whether there is any blood cell component 710b in error sensing portion 760. Thus, it is possible to confirm whether the prescribed amount of blood cell component 710b has been measured or not at blood cell measuring portion 103.

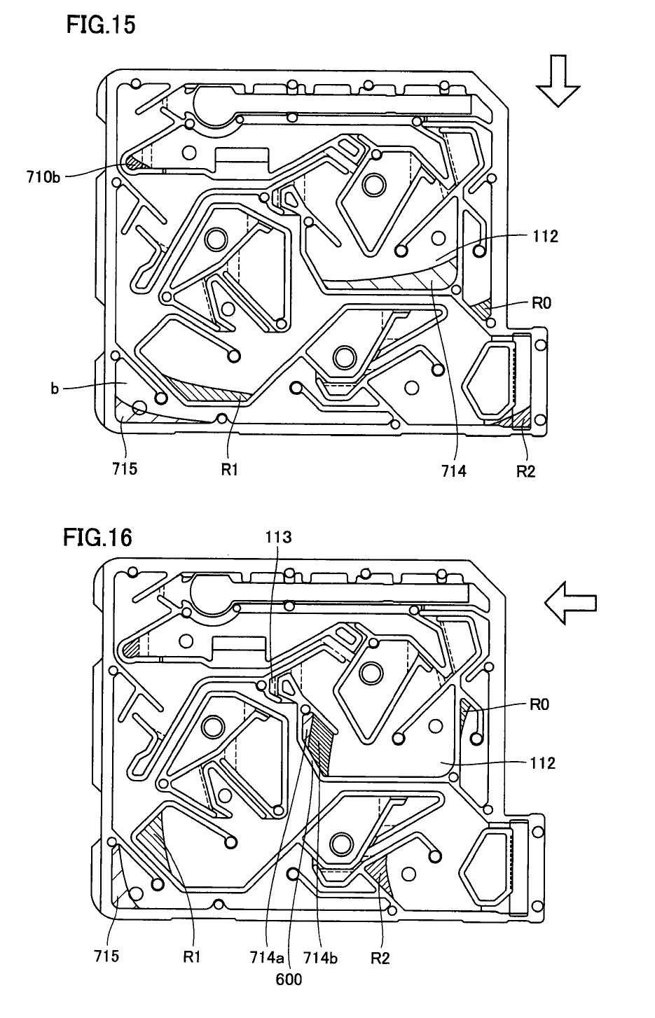

Next, as shown in FIG. 15, by applying centrifugal force in downward direction of FIG. 15 to microchip 500, primary liquid mixture 714 is further mixed sufficiently at the first mixing portion 112. Further, at an area b of microchip 500, excess liquid 715 accumulates.

Next, as shown in FIG. 16, when centrifugal force to the left of FIG. 16 is applied, primary liquid mixture 714 is centrifuge-separated to residue 714a and supernatant 714b at the residue separation portion 600. At this time, residue 714a of primary liquid mixture 714, which is a solid component, is held on the side of second wall 3 of the approximately V-shaped wall forming residue separating portion 600. As the first wall 2 forming the approximately V-shaped wall has such a length that prevents supernatant 714b of primary liquid mixture 714 from flowing out from residue separating portion 600, leakage of supernatant 714b to liquid mixture measuring portion 113 can be prevented.

Next, as shown in FIG. 17, by applying centrifugal force in upper left direction at a diagonal angle of 45° in FIG. 17, only the supernatant 714b of primary liquid mixture 714 is moved to liquid mixture measuring portion 113. At this time, the first wall 2 forming the approximately V-shaped wall has such a length that prevents residue 714a of primary liquid mixture 714 from flowing out from residue separating portion 600, residue 714a does not leak to liquid mixture measuring portion 113. Further, as the second wall 3 forming the approximately V-shaped wall is bent inward, only the supernatant 714b can be moved smooth to liquid mixture measuring portion 113.

Next, as shown in FIG. 18, by applying centrifugal force to the left of FIG. 18, a prescribed amount of supernatant 714b comes to be held in liquid mixture measuring portion 113.

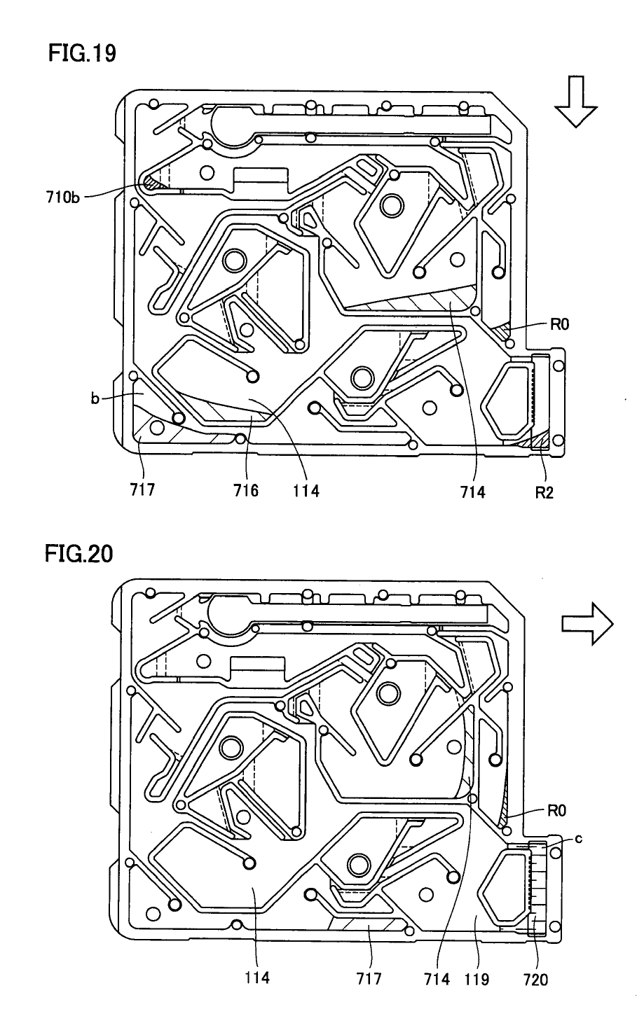

Next, as shown in FIG. 19, by applying centrifugal force in the downward direction of FIG. 19, supernatant 714b moves to the second mixing portion 114, where it is mixed with liquid reagent R1, resulting in secondary liquid mixture 716. Further, excess liquid 717 accumulates in area b of microchip 500.

Finally, as shown in FIG. 20, centrifugal force is applied to the right of FIG. 20, whereby secondary liquid mixture 716 moves to the area c of third mixing portion 119, where it is mixed with liquid reagent R2, resulting in tertiary liquid mixture 720. Tertiary liquid mixture 720 contained in area c (detecting portion) is subjected to optical measurement, in which, for example, area c is irradiated with light and intensity of transmitted light is measured.

Although the present invention has been described and illustrated in detail, it is clearly understood that the same is by way of illustration and example only and is not to be taken by way of limitation, the scope of the present invention being interpreted by the terms of the appended claims,

What is claimed is:

1. A microchip, comprising:
a second substrate, and a first substrate having a groove on its surface, stacked on said second substrate; and
a fluid circuit composed of a cavity defined by said groove and a surface of said second substrate on the side of said first substrate; wherein
said fluid circuit includes a mixing portion as a chamber for mixing two or more liquids; and
at least a part of a wall forming said mixing portion consists of a linearly extending first wall and a second wall linearly extending from an end of said first wall.

2. The microchip according to claim 1, wherein
an inner angle formed by said first and second walls is about 90°.

3. The microchip according to claim 1, wherein
the wall defining said mixing portion includes
a third wall extending from an end of said first wall opposite to the end coupled to said second wall, and
a fourth wall extending from an end of said second wall opposite to the end coupled to said first wall; and
an inner angle formed by said first and third walls and an inner angle formed by said second and fourth walls are each larger than 0° and smaller than 180°.

4. A method of using the microchip according to claim 1, comprising:
the step of introducing two or more liquids to said mixing portion to obtain a liquid mixture by applying centrifugal force to the microchip;
the first mixing promoting step of applying centrifugal force in a direction substantially vertical to said first wall to the microchip; and
the second mixing promoting step of applying centrifugal force in a direction substantially vertical to said second wall to the microchip.

5. The method of using the microchip according to claim 4, wherein
said first and second mixing promoting steps are repeated alternately.

6. A microchip, comprising:
a second substrate, and a first substrate having a groove on its surface, stacked on said second substrate; and
a fluid circuit composed of a cavity defined by said groove and a surface of said second substrate on the side of said first substrate; wherein
said fluid circuit includes
a first mixing portion for mixing a specimen with a first liquid reagent to prepare a primary liquid mixture,
a second mixing portion for mixing said first primary liquid mixture with a second liquid reagent to prepare a secondary liquid mixture, and
a third mixing portion for mixing said secondary liquid mixture with a third liquid reagent to prepare a tertiary liquid mixture;
at least one of said first, second and third mixing portions includes a residue separating portion for separating residue contained in at least one of said primary, secondary and tertiary liquid mixtures; and
said residue separating portion is structured to allow separation of said residue by applying centrifugal force in a first direction to said microchip, and to allow only a supernatant to flow out from said residue separating portion by applying centrifugal force in a second direction.

7. The microchip according to claim 6, wherein
an inner angle θ formed by said first and second directions is 30° to 60° (where θ is an inner angle smaller than 90°).

8. The microchip according to claim 6, wherein
said residue separating portion has an approximately V-shaped wall; and
one of two walls forming said approximately V-shaped wall has such a length that prevents said supernatant and said residue from flowing out from said residue separating portion when centrifugal force in said first direction is applied and allows only said supernatant to flow out from said residue separating portion when centrifugal force in said second direction is applied.

9. The microchip according to claim 6, wherein
said fluid circuit further includes a measuring portion for measuring said supernatant flowing out from said residue separating portion.
